(19) United States
(12) Patent Application Publication
(10) Pub. No.: US 2006/0074431 A1
(43) Pub. Date: Apr. 6, 2006

(54) Disc distraction instrument and measuring device

(75) Inventors: Jeffrey Karl Sutton, Medway, MA (US); Hassan Serhan, South Easton, MA (US); Michael J. O'Neil, West Barnstable, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: DePuy Spine, Inc.

(21) Appl. No.: 10/952,528

(22) Filed: Sep. 28, 2004

(51) Int. Cl. A61B 17/58

(52) U.S. Cl. 606/90

(57) ABSTRACT

A distraction instrument distracts two adjacent vertebra segments to receive an artificial disc, the instrument including a measurement indication related to a distracted disc space. The measurement indication can be the force required to distract the disc space, the distance between the adjacent vertebra, the lordotic angle of a pair of vertebra defining the disc space, and the width of the disc space.

### BACKGROUND OF THE INVENTION

An intervertebral disc has several important functions, including functioning as a spacer, a shock absorber, and a motion unit. The disc maintains the separation distance between adjacent boney vertebral bodies. The separation distance allows motion to occur, with the cumulative effect of each spinal segment yielding the total range of motion of the spine in several directions. Proper spacing is important because it allows the intervertebral foramen to maintain its height, which allows the segmental nerve roots room to exit each spinal level without compression.

Intervertebral discs allow the spine to compress and rebound when the spine is axially loaded during such activities as jumping and running. Importantly, they resist the downward pull of gravity on the head and trunk during prolonged sitting and standing, as well as allow each spinal segment to flex, rotate, and bend to the side, all at the same time during a particular activity. This would be impossible if each spinal segment were locked into a single axis of motion.

An unhealthy disc may result in pain. One way a disc may become unhealthy is when the inner nucleus dehydrates. This results in a narrowing of the disc space and a bulging of the annular ligaments. With progressive nuclear dehydration, the annular fibers can crack and tear. Further, loss of normal soft tissue tension may allow for a partial dislocation of the joint, leading to bone spurs, foraminal narrowing, mechanical instability, and pain.

Lumbar disc, in particular, disease can cause pain and other symptoms in two ways. First, if the annular fibers stretch or rupture, the nuclear material may bulge or herniate and compress neural tissues resulting in leg pain and weakness. This condition is often referred to as a pinched nerve, slipped disc, or herniated disc. This condition will typically cause sciatica, or radiating leg pain as a result of mechanical and/or chemical irritation of the nerve root. Although the overwhelming majority of patients with a herniated disc and sciatica heal without surgery, if surgery is indicated it is generally a decompressive removal of the portion of herniated disc material, such as a discectomy or microdiscectomy. Second, mechanical dysfunction may cause disc degeneration and pain (e.g. degenerative disc disease). For example, the disc may be damaged as the result of some trauma that overloads the capacity of the disc to withstand increased forces passing through it, and inner or outer portions of the annular fibers may tear. These torn fibers may be the focus for inflammatory response when they are subjected to increased stress, and may cause pain directly, or through the compensatory protective spasm of the deep paraspinal muscles. This mechanical pain syndrome, unresponsive to conservative treatment, and disabling to the individuals way of life, is generally the problem to be addressed by spinal fusion or artificial disc technologies.

Traditionally, spinal fusion surgery has been the treatment of choice for individuals who have not found pain relief for chronic back pain through conservative treatment (such as physical therapy, medication, manual manipulation, etc), and have remained disabled from their occupation, from their activities of daily living, or simply from enjoying a relatively pain-free day-to-day existence. While there have been significant advances in spinal fusion devices and surgical techniques, the procedure does not always work reliably.

Artificial discs offer several theoretical benefits over spinal fusion for chronic back pain, including pain reduction and a potential to avoid premature degeneration at adjacent levels of the spine by maintaining normal spinal motion. However, like spinal fusion surgery, distraction instruments and trial spacers are used to distract the intervertebral space and determine a correct size artificial disc or spinal implant. Thus, there remains a need for an improved distraction instrument which distracts the intervertebral space and provides a measurement indiction related to the distracted disc space thereby eliminating the need for trial spacers.

### SUMMARY OF THE INVENTION

The present invention relates generally to a distraction instrument for distracting two adjacent vertebra segments to receive an artificial disc therebetween and provide a measurement indication related to the distracted disc space. The distraction instrument of the present invention has particular application, but is not limited to, direct anterior or oblique-anterior approaches to the spine.

There is provided an intervertebral distraction instrument including a body element having a handle and a pair of diametrically opposing distraction members, and a measurement indicator located on a surface of the distraction instrument. The measurement indicator can provide a distance measurement between the pair of diametrically opposing distraction members. The measurement indicator can also provide a force measurement of the amount force required to distract the pair of diametrically opposing distraction members.

The distraction instrument can include an angulation mechanism for measuring a lordotic angle or a kyphotic angle associated with an intervertebral endplate. The angulation mechanism can include at least one member selected from the group consisting of a hinge and a screw mechanism, a worm and a pinion mechanism, a wedge mechanism, and an elliptical cam mechanism. The rotation end can be a knob. The knob can provide an indication of a lordotic angle or a kyphotic angle associated with an intervertebral endplate.

The diametrically opposed distraction members can include tines having a width mechanism for changing the distance between the tines. The width mechanism can include at least one member selected from the group consisting of a rack and a pinion mechanism, a link and a slide mechanism, and a wedge mechanism. The distraction members can also include a force sensor located on vertebral endplate engagement surfaces.

The present invention has many advantages, such as measuring a vertical and a horizontal distance of the disc space, measuring the lordotic angle or the kyphotic angle of the disc space, and measure forces related to the distraction process. All these advantages can be used to determine the correct sized artificial disc, thereby eliminating a step in previous procedures.

### BRIEF DESCRIPTION OF THE DRAWINGS

FIG. 1 shows distraction instrument according to the principals of the present invention;

FIG. 2A shows distraction instrument of FIG. 1 in a contracted position;

FIG. 2B shows distraction instrument of FIG. 1 in a distracted position;

FIG. 3A shows an embodiment of angulation mechanism having a screw mechanism and a hinge mechanism;

FIG. 3B shows another embodiment of the fixed scale and pointer of FIG. 3A;

FIG. 4A shows another embodiment of the angulation mechanism of FIG. 3A having a worm gear and a pinion gear;

FIG. 4B shows another embodiment of the angulation mechanism of FIG. 3A having a wedge mechanism;

FIG. 4C shows another embodiment of the angulation mechanism of FIG. 3A having a elliptical cam mechanism;

FIG. 4D shows another embodiment of the angulation mechanism of FIG. 3A having a worm/pinion gear mechanism;

FIG. 4E shows the angulation mechanism of FIG. 4D in its maximum and minimum position;

FIG. 5 shows another embodiment of the distraction instrument of FIG. 1;

FIG. 6 shows another embodiment of the distraction members of the preceding figures;

FIG. 7A shows an embodiment of a width mechanism having a rack and pinion gear;

FIG. 7B shows another embodiment of a width mechanism having a slide/link width mechanism;

FIG. 7C shows another embodiment of a width mechanism having a slide/wedge width mechanism;

FIG. 8 shows a force and displacement measurement mechanism; and

FIG. 9 shows a perspective view of an embodiment of distraction instrument.

### DETAILED DESCRIPTION OF THE INVENTION

The foregoing and other objects, features and advantages of the invention will be apparent from the following more particular description of preferred embodiments of the invention, as illustrated in the accompanying drawings in which like reference characters refer to the same parts throughout the different views. The same number appearing in different drawings represents the same item. The drawings are not necessarily to scale, with emphasis instead being placed upon illustrating the principles of the invention.

In general, the surgical procedure of the present invention for implantation utilizes an anterior approach. During the surgery, a small incision is made in the abdomen below the belly button. The organs are carefully moved to the side so the surgeon can visualize the spine. The surgeon then removes a portion of a disc creating a disc space. The disc space is distracted using a distraction instrument and a size of an implant is determined. In one embodiment, the implant is inserted; endplates first followed by the polyethylene core. In another embodiment, the entire implant assembly (e.g., both prosthetic endplates and its core) is inserted simultaneously. The implant stays in place from the tension in spinal ligaments and the remaining part of the annulus of the disc. In addition, compressive forces of the spine keep the implant in place. A successful implantation is governed by good patient selection, correct artificial disc size selection, and proper artificial disc positioning. A distraction instrument which distracts the disc space and provides measurement information related to the disc space can be used to chose the correct artificial disc size.

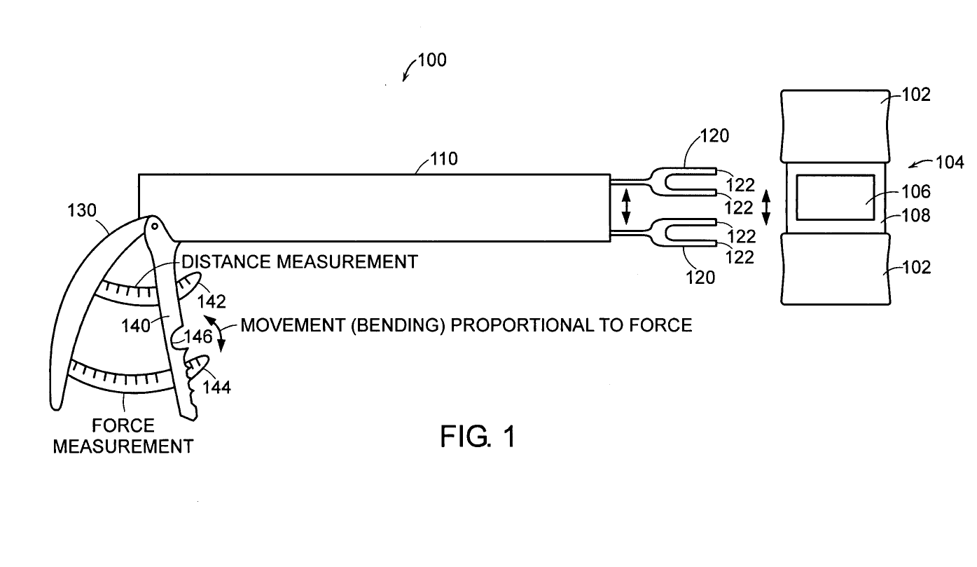

FIG. 1 shows distraction instrument 100, vertebral bodies 102, and damaged disc 104. The distraction instrument 100 includes body element 110, distraction members 120 having distraction tines 122, handle 130, trigger mechanism 140, distance measurement indicator 142, and force measurement indicator 144.

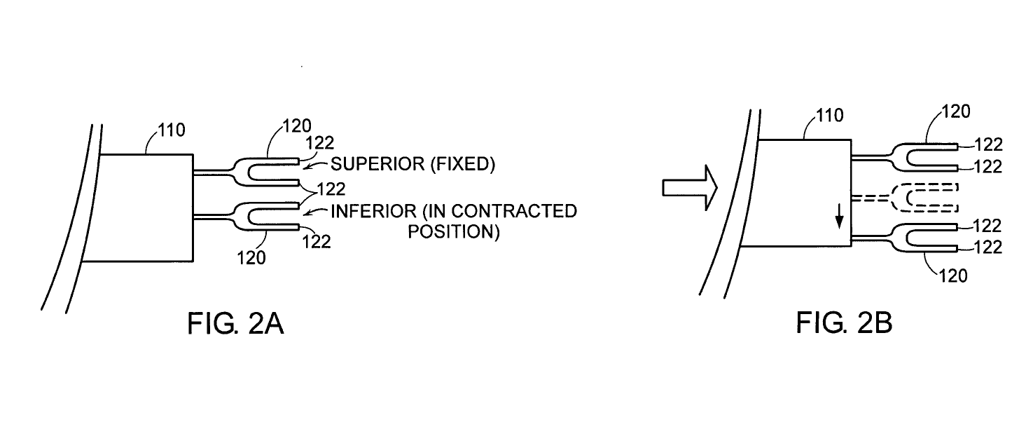

The damaged disc 104 is prepared to receive an artificial disc by removing window 106 the width of the artificial disc to be implanted from annulus 108 of damaged disc 104. Since annulus 108 is a fibrous material it is desirable not to over distract the disc space causing annulus 108 to tear. The nucleus pulposus of disc 104 is completely removed. The distraction members 120 of distraction instrument 100 are inserted into window 106 of damaged disc 104 in a contracted position as shown in FIG. 2A. The trigger mechanism 140 is then drawn toward handle 130 causing the distraction members 120 to become displaced from the contracted position (FIG. 2A) to a distracted position (FIG. 2B). One distraction member 120 may be fixed to body element 110 allowing the other distraction member to move relative to the fixed distraction member thereby creating the distraction. However, both distraction members 120 may be movable with relation to body element 110 thereby creating the distraction.

Distraction of the disc space may occur multiple times during the implantation procedure. To that end, the amount of force required to distract the disc space can be recorded using force measurement indicator 144 allowing the procedure to be substantially repeated without damaging annulus 108. The distance of the distracted space may also be recorded using distance measurement indicator 142. The recorded distance can be helpful in choosing the height of the artificial implant. Although measurement indicators 142, 144 are shown on handle 130 they can be anywhere on the distraction instrument 100.

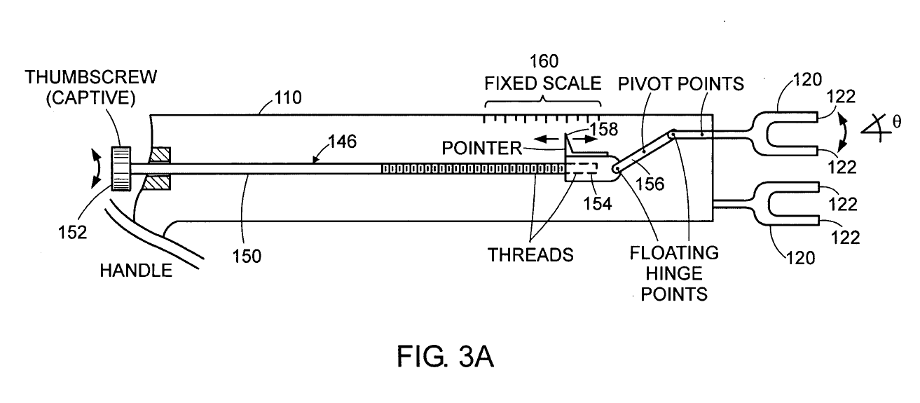

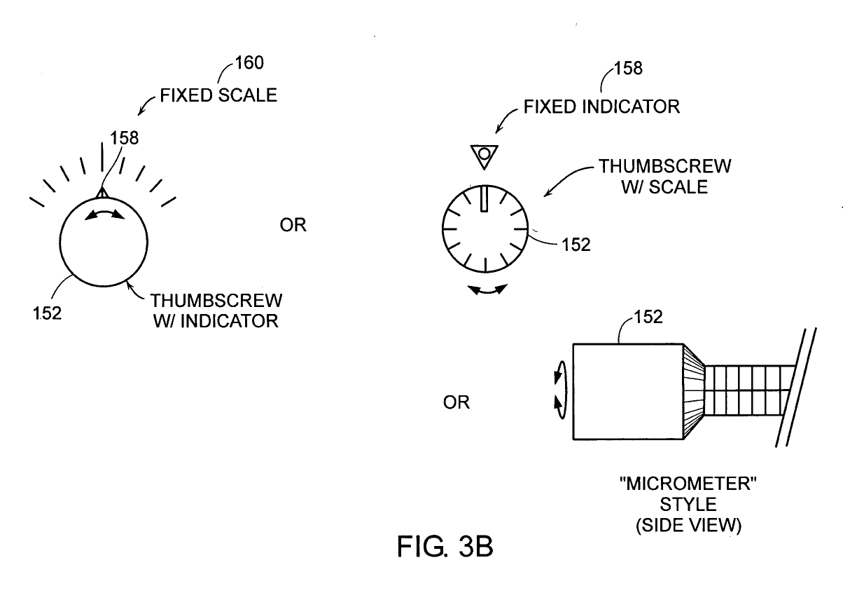

FIG. 3A shows angulation mechanism 146 of another embodiment of the invention. The angulation mechanism 146 includes shaft 150, knob or thumbscrew 152, screw mechanism 154, hinge mechanism 156, and pointer 158. In another embodiment, thumbscrew 152 can be adapted to allow for increased torque, such as including a socket for a torque wrench or the like. The angulation mechanism 146 is pivotally attached to distraction member 120 to provide and indication of the lordotic angle or the kyphotic angle of the vertebral endplate. Rotation of thumbscrew 152 causes more or less thread engagement with screw mechanism 146, causing distraction member 120 to angulate about its pivot point. Linear displacement of pointer 158 can be read against fixed scale 160 to determine the lordotic angle or the kyphotic angle.

FIG. 3B shows another embodiment of pointer 158 and fixed scale 160. The pointer 158 and fixed scale are located on thumbscrew 152 and body element 110. This location may be desirable over the location shown in FIG. 3A since the deployed end of the instrument 100 is in the spine of the patient and may be difficult to observe the indicator mark and/or the scale.

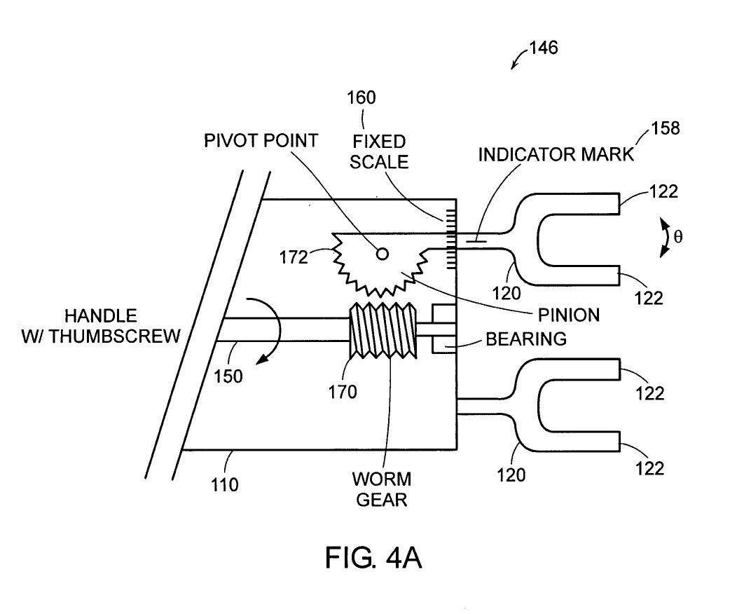

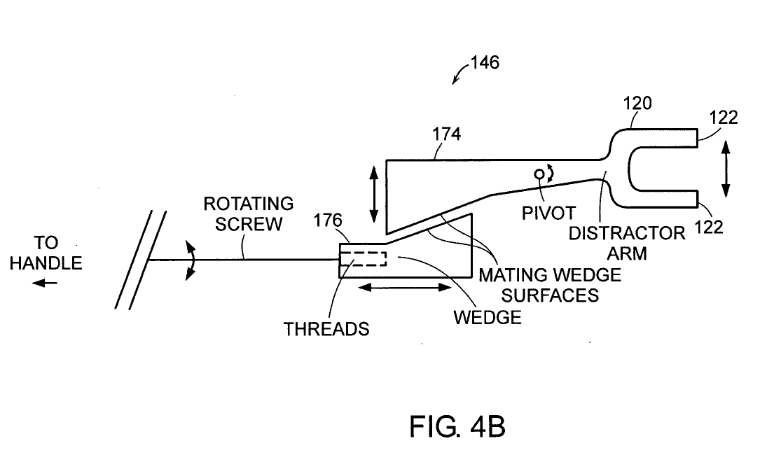

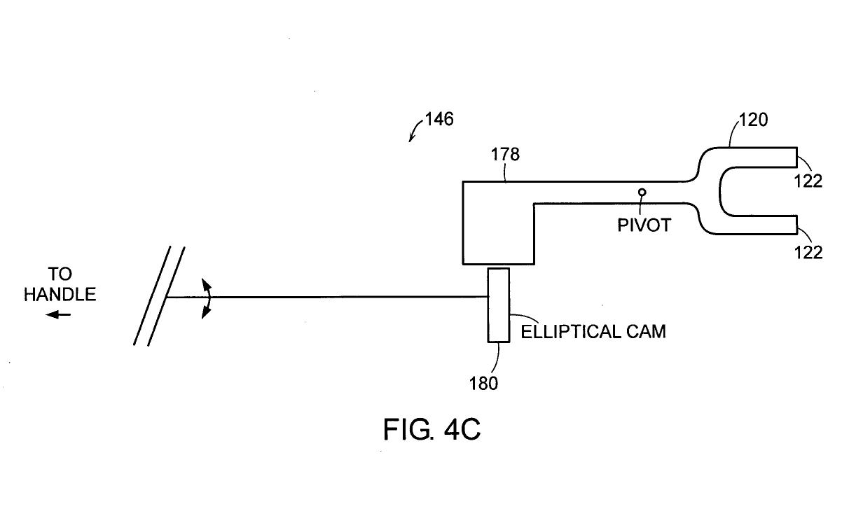

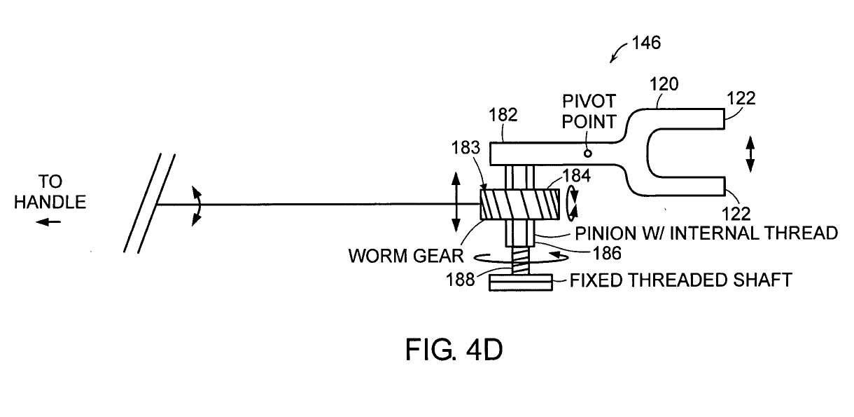

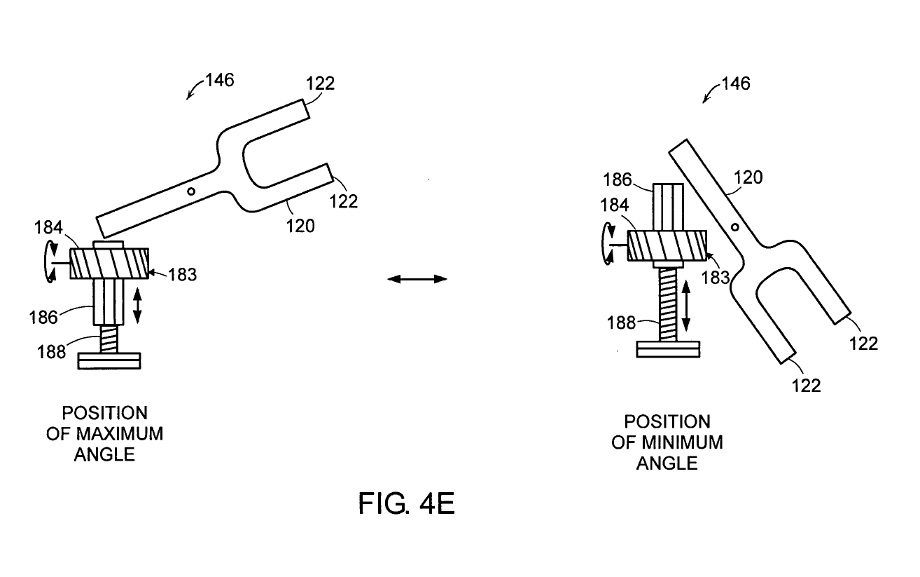

FIGS. 4A-4E show alternative embodiments of angulation mechanism 146 of FIG. 3A. More specifically, screw mechanism 154 and hinge mechanism 156 can be replaced by worm gear 170 and pinion gear 172 (FIG. 4A), wedges 174, 176 (FIG. 4B), mating surface 178 and elliptical cam 180 (FIG. 4C), or worm/pinion gear 183 (FIGS. 4D and 4E).

FIG. 4A shows angulation mechanism 146 having worm gear 170 and pinion gear 172. The pointer or indicator 158 can be located on distraction member 120 and fixed scale 160 can be located on a distal end of body element 110. Rotation of thumbscrew 152 (FIGS. 3A and 3B) causes more or less worm and pinion engagement, causing distraction member 120 to angulate about its pivot point. Linear displacement of indicator 158 can be read against fixed scale 160 to determine the lordotic angle or the kyphotic angle.

FIG. 4B shows angulation mechanism 146 having wedges 174, 176. Rotation of thumbscrew 152 (FIGS. 3A and 3B) causes more or less thread engagement in wedge 176, causing distraction member 120 to angulate about its pivot point.

FIG. 4C shows angulation mechanism 146 having mating surface 178 and elliptical cam 180. Rotation of thumbscrew 152 (FIGS. 3A and 3B) causes elliptical cam 170 to engage mating surface 178 thereby changing the angle of distractor mechanism 120. The use of a simple cam may not have sufficient strength to withstand the forces imposed during distraction, but could be embellished with a reduction gear train and/or a ratchet and pawl mechanism to ensure that the distraction forces do not overcome the cam position.

FIGS. 4D-4E shows angulation mechanism 146 having worm/pinion gear 183. A fixed threaded rod 188 is fixedly coupled body element 110 (FIG. 1). Rotation of thumbscrew 152 (FIGS. 3A and 3B) rotates worm gear 184, which in turn rotates and drives pinion gear 184. The pinion gear 184 has an internal thread and is mounted on a fixed threaded rod 188, such that as pinion gear 184 rotates it experiences a linear displacement proportional to the thread pitch of fixed threaded rod 188. Such a mechanism would be robust and be able to withstand forces involved with distraction (if suitably sized), since worm gears resist backlash from loads. It would be desirable that pinion gear 184 not contact and wear on distractor mechanism 120 directly, but rather be connected by a yoke, thrust washer, or other suitable sliding mechanism (not shown).

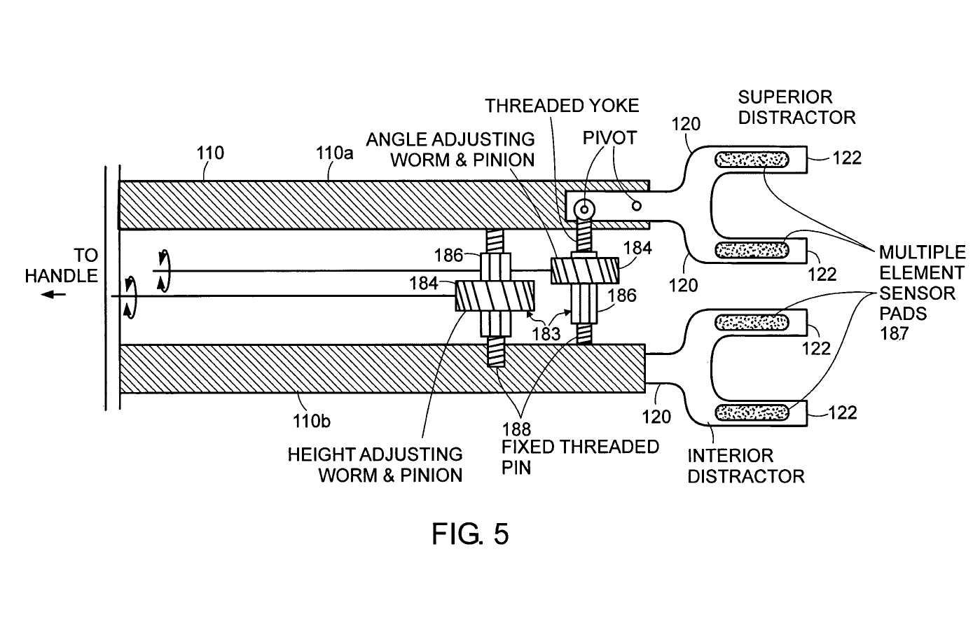

FIG. 5 shows another embodiment of the present invention having a pair of worm/pinion gears 183, one used for distraction and the other used for angulation. Such an embodiment would be able to deliver more force than a simple pliers mechanism. The force could be measured by the torque required to turn the worm gear or by using force sensors 187 on the surfaces of distractor members 120. The force sensors could communicate if the distraction forces are being evenly distributed across the disc space. Thus, a surgeon would have infinite adjustment capability with respect to the height and angle. The height and angle adjustment can work jointly or independently. To work independently, fixed threaded pin 188 can be attached to an extension of superior distractor body 110a, rather than fixed to the inferior distractor body 110b.

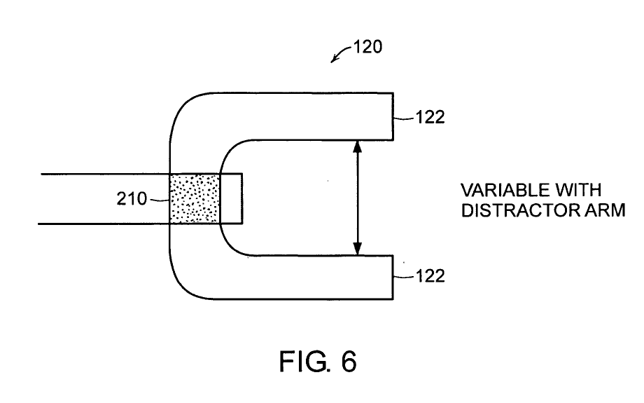

FIG. 6 shows another embodiment of distraction members 120 of the preceding figures. The distraction member 120 includes a width mechanism 210 for varying the width of distractor tines 122. This allows for the distraction forces to be spread as close to the cortical bone as possible, and not concentrated in the center of the intervertebral endplates. Also, it allows for a universal distraction instrument, rather than requiring a specific one for each different size implant. Further, it can provide a quantitive measurement for “border line” size approximations.

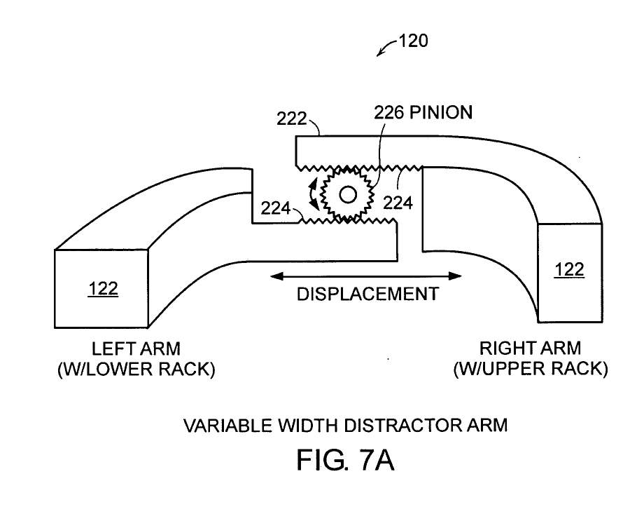

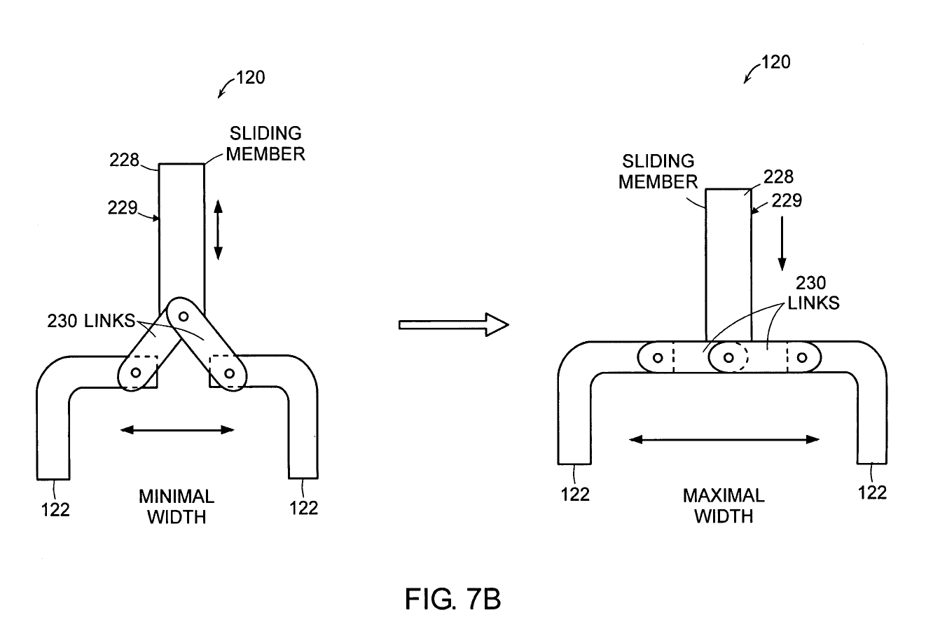

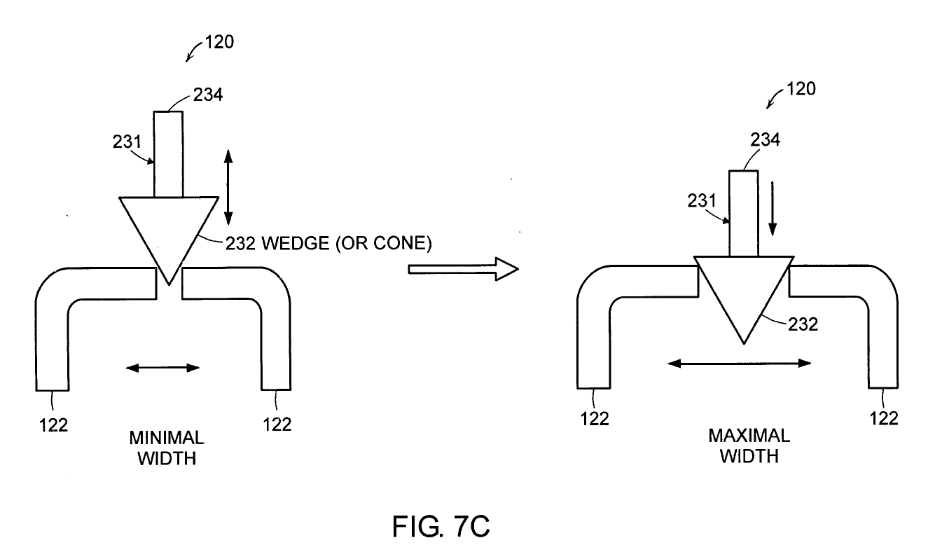

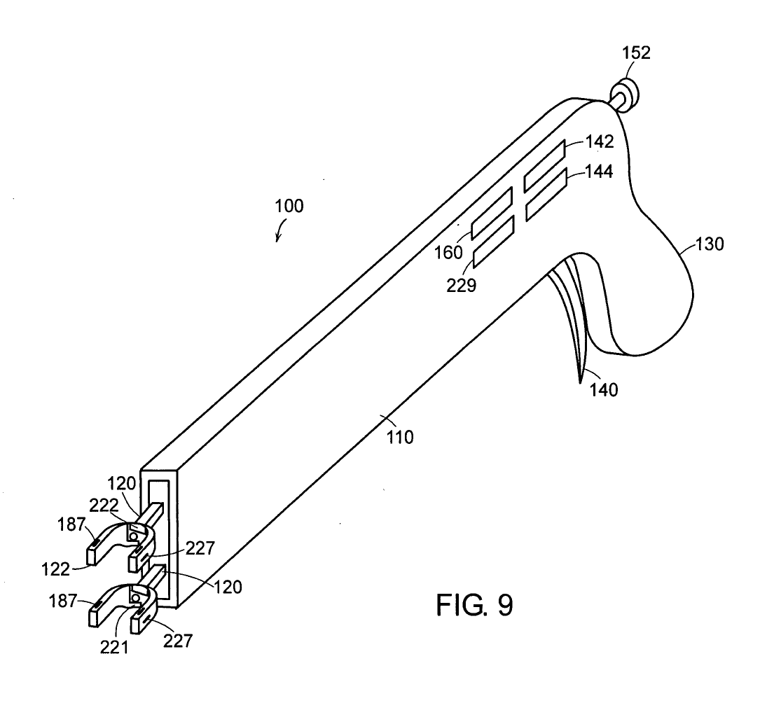

FIG. 7A shows a rack and pinion gear 222 used as width mechanism 210 of FIG. 6. Each tine 122 includes rack gear 224 riding on drive pinion 226. One tine 122 would be an upper rack and the other tine 122 would have a lower rack, such that when the pinion is rotated both tine 122 move either towards each other or away from each other, depending on the direction of pinion rotation. The distance could be measured according the amount of rotation need to extend tine 122. Further, the forces involved in expanding the width of the distractor member 120 could be measured from the torque required to rotate pinion 226, or from force sensors (227 FIG. 9) located on external edges of tines 122.

FIGS. 7B and 7C shows other embodiments of width mechanism 210 of FIG. 6. More specifically, FIG. 7B shows slide/link width mechanism 229 having slide member 228 and link member 230 used to extend or retract tines 122 by moving slide member 228 toward of away from link member 330. FIG. 7C shows wedge/slide width mechanism 231 having wedge or cone member 232 and slide member 234 used to extend or retract tines 122 by moving slide member 234 toward of away from wedge or cone member 232. The width mechanisms 229, 231 of FIGS. 7B and 7C could provide a user with tactile feedback of the force required for displacement, as well as a simple gauge or scale to measure the displacement. The mechanisms could also use a ratchet and pawl mechanism (not shown) to hold the adjustment in place, as well as a spring to maintain closure/contact with wedge or cone 232.

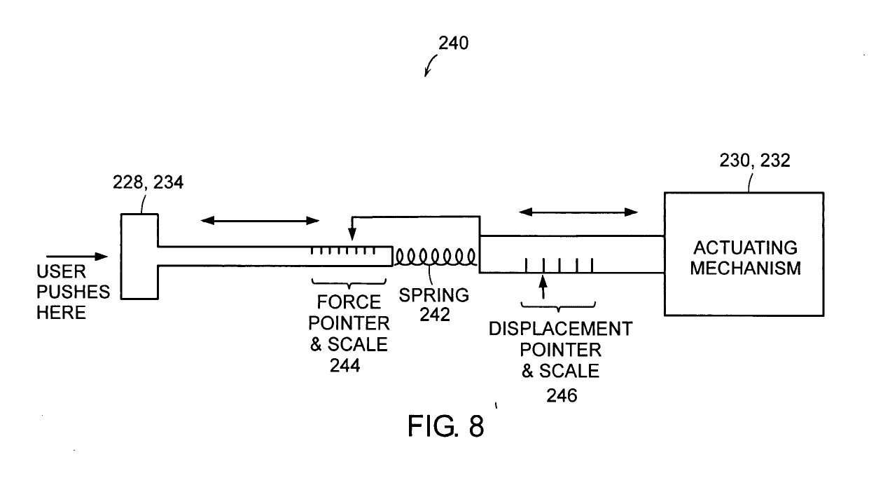

FIG. 8 shows force and displacement measurement mechanism 240 using the width mechanisms 229, 231 of FIGS. 7B and 7C. Similar to multi-function trigger mechanism 140 (FIG. 1), force and displacement can be measured on slide member 228, 234 by the incorporating of spring 241 between slide member 228 and link member 230 or slide member 234 and wedge member 232. The displacement measurement mechanism 240 could use a force pointer and scale 244 and displacement pointer and scale 246 to show the amount of force and displacement required. The force pointer 244 could be rigidly fixed to a moving distal portion of mechanism 240 so that the force measurements are independent of linear displacements.

FIG. 9 shows a perspective view of an embodiment of distraction instrument 100 including body element 110, distraction members 120 having distraction tines 122, handle 130, trigger mechanism 140, thumbscrew 152, distance measurement indicator 142, force measurement indicator 144, lordotic/kyphotic angle indicator 160, and width measurement indicator 229. The distraction members 120 include force sensors 187, 227, and rack and pinion gear 222. The indicators 142, 144, 160, and 229 may be a digital display. The distraction instrument 100 can include an input/output port to provide data to a computer for further processing. Operation of the distraction instrument 100 can be in accordance with the reference to the description of the preceding figures.

While this invention has been particularly shown and described with references to preferred embodiments thereof, it will be understood by those skilled in the art that various changes in form and details may be made therein without departing from the scope of the invention encompassed by the appended claims.

What is claimed is:

1. An intervertebral distraction instrument, comprising:
a body element having a proximal end and a distal end;
a pair of diametrically opposing distraction members movably coupled to the distal end of the body element, wherein one distraction member is fixed to the distal end of the body element and the other distraction member is movable relative to the fixed distraction member;
a handle coupled to the proximal end of the body element; and
a measurement indicator located on a surface of the distraction instrument.

2. The instrument of claim 1, wherein the measurement indicator provides a distance measurement between the pair of diametrically opposing distraction members.

3. The instrument of claim 1, wherein the measurement indicator provides a force measurement relative to an amount of force required to distract the pair of diametrically opposing distraction members.

4. The instrument of claim 1, wherein the handle includes a movably coupled trigger mechanism for distracting the pair of diametrically opposing distraction members.

5. The instrument of claim 1, further comprising an angulation mechanism having a connection end and a rotation end, the connection end coupled to one distraction member for measuring an angle associated with an intervertebral endplate.

6. The instrument of claim 5, wherein the angulation mechanism includes at least one member selected from the group consisting of a hinge and a screw mechanism, a worm and a pinion mechanism, a wedge mechanism, and an elliptical cam mechanism.

7. The instrument of claim 6, wherein the rotation end includes a knob.

8. The instrument of claim 7, wherein the knob provides an indication of a lordotic angle or a kyphotic angle associated with an intervertebral endplate.

9. The instrument of claim 1, wherein a distraction member has a proximal end and a distal end, the distal end including tines and the proximal end including a width mechanism for changing the distance between the tines.

10. The instrument of claim 11, wherein the width mechanism includes at least one member selected from the group consisting of a rack and a pinion mechanism, a link and a slide mechanism, and a wedge mechanism.

11. The instrument of claim 1, wherein the distraction members include at least one force sensor located on vertebral endplate engagement surfaces.

12. A method of measuring distance and force related to distracting an intervertebral disc space, comprising:
preparing an intervertebral disc space for distraction;
inserting a distraction instrument into the intervertebral disc space;
distracting a movable distraction member relative to a fixed distraction member thereby distracting the intervertebral disc space; and
measuring a component related to distracting the intervertebral disc space.

13. The method of claim 12, wherein the component related to distracting the intervertebral disc space is a force component.

14. The method of claim 13, wherein the force component is an amount of force required to distract the disc space with the distraction instrument.

15. The method of claim 13, wherein the force component is an amount of force required to extend distraction tines of the distraction instrument within the intervertebral disc space.

16. The method of claim 12, wherein the component related to distracting the intervertebral disc space is a distance component.

17. The method of claim 16, wherein the distance component is an amount of distance between adjacent vertebra.

18. The method of claim 16, wherein the distance component is an amount of distance required to extend distraction tines of the distraction instrument to an annulus of the prepared disc.

19. The method of claim 12, wherein the component related to distracting the intervertebral disc space is an angular component.

20. The method of claim 19, wherein the angular component is a lordotic angle or a kyphotic angle of an intervertebral disc.

21. An intervertebral distraction instrument, comprising:
a pair of diametrically opposing distraction members movably coupled to a distal end of a body element, wherein one distraction member is fixed to the distal end of the body element and the other distraction member is movable relative to the fixed distraction member, the distraction members including means for distracting an intervertebral disc space; and
means for measuring a component generated by the means for distracting.
